(19) United States
(12) Patent Application Publication
(10) Pub. No.: US 2016/0129137 A1
(43) Pub. Date: May 12, 2016

(54) COMPOSITION FOR MONITORING AGED TISSUES, COMPRISING BACTERIA, AND USE THEREOF

(71) Applicants: Samsung Electronics Co., Ltd., Suwon-si (KR); Industry Foundation of Chonnam National University, Gwangju (KR)

(72) Inventors: Byounggon Moon, Hwaseong-si (KR); Hyon E. Choy, Seoul (KR); Sungchun Cho, Hwaseong-si (KR); Sangchul Park, Seongnam-si (KR); Jaeho Jeong, Gwangju (KR)

(21) Appl. No.: 14/934,446

(22) Filed: Nov. 6, 2015

(30) Foreign Application Priority Data
Nov. 6, 2014 (KR) 10-2014-0153740

(51) Int. Cl. A61K 49/00

(52) U.S. Cl. CPC A61K 49/0097; A61K 49/0045

(57) ABSTRACT

A recombinant bacterium for detecting aged tissues and a method of detecting aged tissues in a subject, and a composition for delivering a drug to aged tissues are provided.

### RELATED APPLICATION

This application claims the benefit of Korean Patent Application No. 10-2014-0153740, filed on Nov. 6, 2014, in the Korean Intellectual Property Office, the entire disclosure of which is hereby incorporated by reference.

### BACKGROUND

1. Field

The present disclosure relates to recombinant bacteria for detecting aged tissues, methods of detecting aged tissues of a subject, and recombinant bacteria for delivering a drug to aged tissues.

2. Description of the Related Art

Senescent cells have a large flat cell shape, can no longer grow, and secrete a variety of inflammatory proteins. Biochemically, aging is closely related to an increase in the expression of p16 and p21, known as cellular senescence-associated genes, or an increase in the expression of γ-H2A, associated with oxidative damage of DNA.

Cellular senescence and tissue aging can be induced by various factors. During aging, numerous alterations occur in multiple tissues of the body, leading to impaired function, increased vulnerability, and eventually death.

Some bacterial species tend to proliferate and accumulate in tumor cells. Since bacteria are capable of delivering and expressing a variety of therapeutic proteins and are easily eradicated by antibiotic treatment, bacteria have been widely used to treat tumors.

However, use of bacteria to detect aged tissues has not been reported.

### SUMMARY

Provided is a recombinant bacterium for detecting aged tissue in a subject. The recombinant bacterium comprises a mutation in a relA gene that reduces relA activity as compared to the same bacterium without the mutation, a mutation in a spoT gene that reduces spoT activity as compared to the same bacterium without the mutation, or a combination thereof. The recombinant bacterium further comprises a reporter gene encoding a fluorescent protein or a luminescent protein.

Also provided are methods of detecting aged tissue in a subject. The methods comprise administering to the subject the recombinant bacterium and detecting a position and/or intensity of a signal generated by the reporter gene in the subject, wherein position of the signal, or the position of the greatest intensity of the signal, corresponds to a location of aged tissue in a subject.

Further provided are compositions for delivering a drug to an aged tissue. The compositions comprise the recombinant bacterium and a drug contained within the bacterium and/or produced by the bacterium.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and/or other aspects will become apparent and more readily appreciated from the following description of the exemplary embodiments, taken in conjunction with the accompanying drawings in which:

FIG. 1A illustrates results of (a) in vitro and (b) in vivo IVIS imaging of the SHJ2168 strain.

FIG. 1B illustrates a correlation between the amount of the SHJ2168 strain injected and the intensity of the detected signals.

FIG. 2A provides images showing targeting of the SHJ2168 strain injected into young mice and aged mice.

FIG. 2B provides images showing targeting of the SHJ2168 strain injected into mice with various ages.

FIG. 3 provides images showing targeting of the SHJ2168 strain injected into 20-month old mice; and

FIG. 4 illustrates a relationship between expression levels of p16 and γH2A and targeting of the SHJ2168 strain in various sections of ileum of 24-month old mice.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Provided herein is a recombinant bacteria useful for monitoring an aged tissue, the bacteria comprising a mutant relA gene, a mutant spoT gene, or a combination thereof, and a reporter gene, as well as a method for monitoring or detecting an aged tissue using the bacteria.

The term “reporter gene” may refer to a polynucleotide encoding a fluorescent protein, a luminescent protein, or the like. The fluorescent protein may be a green fluorescent protein, a blue fluorescent protein, a yellow fluorescent protein, a red fluorescent protein, or any combination thereof. The luminescent protein may be firefly luciferase, Renilla luciferase, Metridia luciferase, bacteria luciferase, or any combination thereof. The luciferase may be derived from Vibrio harveyi, V. fischeri, Photobacterium phosphoreum, P. leiognathi, or P. luminescens. The reporter gene may be present in a vehicle (e.g., expression vector) introduced into the bacteria or the reporter gene may be integrated into a chromosome of the bacteria. Methods of introducing exogenous reporter genes into bacteria are routine and well known to persons of ordinary skill in the art.

The term “recombinant bacterium” refers to a bacterium that has been genetically modified. The bacteria may be Salmonella, Clostridium, or Escherichia genus. The bacteria may be Salmonella typhimurium, S. choleraesuis, or S. enteritidis, E. coli, or any combination thereof.

The relA and spoT genes are genes that encode ppGpp synthetase involved in ppGpp synthesis. Mutation of the relA and/or spoT genes may cause a reduction in transcription and/or translation of the relA and/or spoT genes or a reduction in the activity of proteins encoded thereby. A reduction in gene activity may indicate that no products are produced from the gene or that a less amount of products is produced as compared with those produced from a non-mutant gene. Thus, a mutation in a relA and/or spoT gene may result in the loss of ppGpp synthesis in a recombinant bacterium, thereby rendering the bacterium non-pathogenic. The mutation may include substitutions, insertions, deletions, or any combination thereof occurring in the entire or partial coding or regulatory sequence of the relA gene, the spoT gene, or any combination thereof. The mutation may be obtained via various mutagenesis methods well known in the art. For example, a deletion of relA gene or spoT gene may be performed by PCR mutagenesis method or cassette mutagenesis method.

The term “aged tissue” refers to a tissue with cells (i.e., aged cells) that have a reduced ability to proliferate, an increase in accumulation of lipofuscin, an increase in the activity of β-galactosidase, an increase in mitochondrial reactive oxygen species, a decrease in mitochondrial membrane potentials, an increase in p16 expression, an increase in γH2A expression, and/or an increase in duration of G0 and/or G1 phase of the cells, as compared with reference cells. The reference cell refers to a cell of the same type, derived from a tissue of a young subject. The young subject may be a mouse aged 4 to 12 weeks, 4 to 8 weeks, or 4 to 6 weeks who is normal and healthy, or a person aged 18 to 25, 18 to 23, or 18 to 20 who is normal and healthy. The ability of a cell to proliferate may be identified by measuring the doubling time of the cell. When the doubling time of a cell is over 14 days, the cell may be referred to as an aged cell. The aged tissues may include a tissue of at least one selected from the group consisting of brain, heart, liver, spleen, intestine, kidney, bladder, skin, muscle, joint, lymphatic gland, and genital organ.

Aged tissues may be monitored or detected by administering the recombinant bacteria to tissue or subject comprising tissue and detecting a position and/or an intensity of a signal output from the fluorescent protein or the luminescent protein generated by the reporter gene of the bacteria administered to tissue or subject. Thus, the method of monitoring (or detecting) aged tissue in a subject may comprise administering the bacteria to a subject and detecting a signal generated by the reporter gene in the subject More specifically, the position of the signal or the position of the greatest intensity of the signal indicates a location of aged tissue in the subject. Thus, the position of the signal can be used to detect or locate aged tissue in the subject. Also, the intensity of the signal will increase according to increasing age of the tissue, increasing concentration of aged cells in the tissue, increasing amount of aged tissue, etc. Thus, changes in the aging of tissue can be monitored by measuring a position or intensity of a signal at a first point in time after administration of the bacteria to the subject, measuring the position or intensity of a signal at a second point in time after administration of the bacteria to the subject, and comparing the position and/or intensity of the signal at the first and second time points. Changes in the position and/or intensity of the signal at the first and second time points correspond to changes in the aging of the tissues.

The bacteria may be in a composition comprising a suitable carrier, such as a buffer solution providing a pH suitable for bacterial survival.

The term “subject” may refer to a mammal. For example, the subject may be a human, mouse, cow, horse, pig, dog, sheep, goat, or cat. When the subject is a mouse, the subject may be over 12-month old, over 18-month old, over 20-month old, over 22-month old, or over 24-month old. When the subject is a human, the subject may be over 43-year old, over 56-year old, over 60-year old, 65-year old, or over 69-year old.

The recombinant bacteria, optionally in a carrier, may be administered by any suitable method. The administering may be performed by a systemic administration. The systemic adminstration may be enteral administration or parenteral administration. The parenteral administration may be performed via intravenous administration, subcutaneous administration, intramuscular administration, or body cavity administration (intraperitoneal, intra-articular, or optical cavity administration).

The recombinant bacterium may be administered intravenously and delivered to a target region such as aged tissues via bloodstream. Dosage may variously be prescribed according to factors such as formulation method, administration method, age, weight, sex, and pathological condition of a patient, administration duration, administration interval, administration route, excretion rate, and reaction sensitivity. The amount of bacteria administered to the subject may be in a range of about 1×10⁵ colony forming units (CFU)/kg to about 1×10¹⁰ CFU/kg, about 1×10⁵ CFU/kg to about 5×10⁹ CFU/kg, or about 1×10⁵ CFU/kg to about 2.5×10⁹ CFU/kg. When the subject is a mouse, the administered amounts of bacteria may be in a range of about 10⁴ to about 10¹⁰, about 10⁵ to about 10⁹, or about 10⁶ to about 10⁸ CFU.

The term “signal” may refer to a signal directly generated by the fluorescent protein or luminescent protein, e.g., upon illumination with activating radiation. The signal may be detected by measuring fluorescence or luminescence. Aged tissues may be detected by identifying a position and/or intensity of the signal.

The recombinant bacteria or composition comprising same is also useful for delivering a drug to aged tissues, wherein the recombinant bacteria includes a reporter gene, bacteria including a mutant relA gene, a mutant spoT gene, or a combination thereof, and a drug. Thus, also provided herein is a method of delivering a drug to aged tissues by administering the recombinant bacteria comprising the drug to a subject.

All aspects of the recombinant bacteria and aged tissues are as described above.

The drug may be delivered to the aged tissues by the bacteria. The drug may be directly contained in the bacteria or produced by metabolism of the bacteria. The drug may be entrapped in nanoparticles or microparticles attached to or contained within the bacteria. If the drug is produced by the metabolism of the bacteria, the bacteria may be a bacteria genetically modified to produce the drug.

The drug may include a compound, peptide, hexane, carbohydrate, lipid, or any combination thereof. The drug may be an anti-aging substance. The drug may be an siRNA or shRNA that suppresses or retards aging by altering expression of a target gene or genes known to affect aging.

Embodiments of the present disclosure will be described in further detail with reference to the following examples. These examples are for illustrative purposes only and are not intended to limit the scope of the present disclosure.

### Example 1: Targeting of Salmonella Strain in Animal Model

1.1. Imaging of Salmonella Strain

Salmonella typhimurium strain SHJ2037 having deficiency in producing a pathogenic regulatory factor of Salmonella, ppGpp, due to deletions of relA and spoT gene (i.e., relA::cat, spoT::kan) has been previously prepared by the present inventors and deposited under accession number of KCTC 10787BP in the Korean Collection for Type Culture in Korea Research Institute of Bioscience and Biotechnology, which is an International Depository Authority. Salmonellae were grown in LB medium containing 50 μg/ml of kanamycin with vigorous aeration at 37° C. For bioluminescence imaging, the bacterial luciferase (lux) operon from S. typhimurium-Xen26 (Xenogen-Caliper) was chromosomally integrated into the strain SHJ2037 by P22HT int transduction. The lux operon encode all the proteins (i.e., luciferase, substrate, and substrate regenerating enzymes) necessary to generate bioluminescence. The resulting Salmonella strain is named SHJ2168.

8-week old male C57BL/6J mice (16-18 g, Samtaco, Gyeonggi-do, Korea) were used in experiments. Animal care, all experiments and euthanasia were performed in accordance with protocols approved by the Chonnam National University Animal Research Committee and the Guide for the Care and Use of Laboratory Animals published by the National Institutes of Health (NIH publication 85-23, revised 1985).

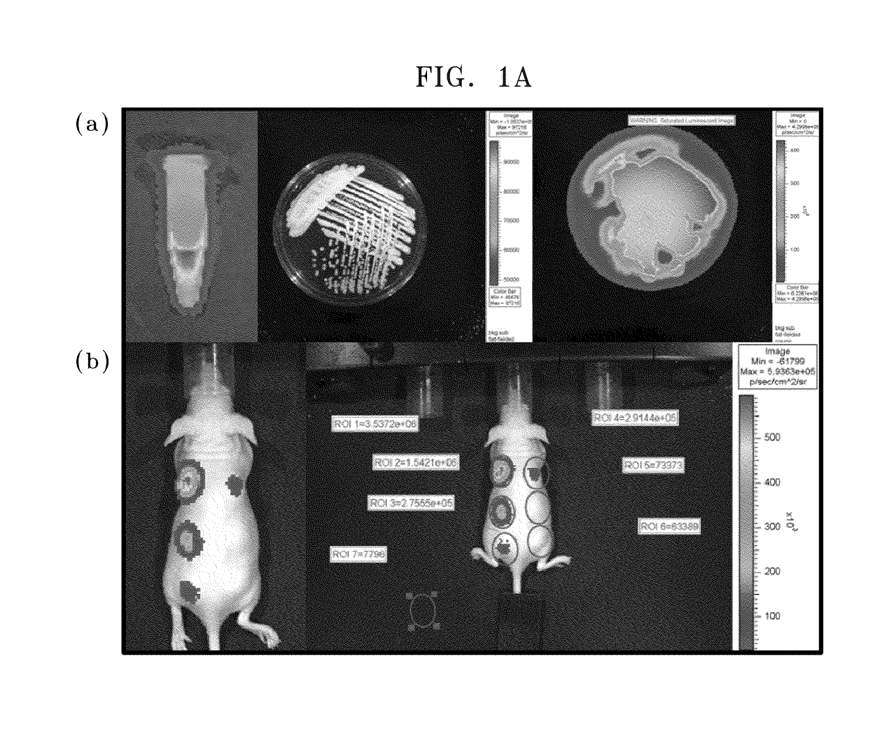

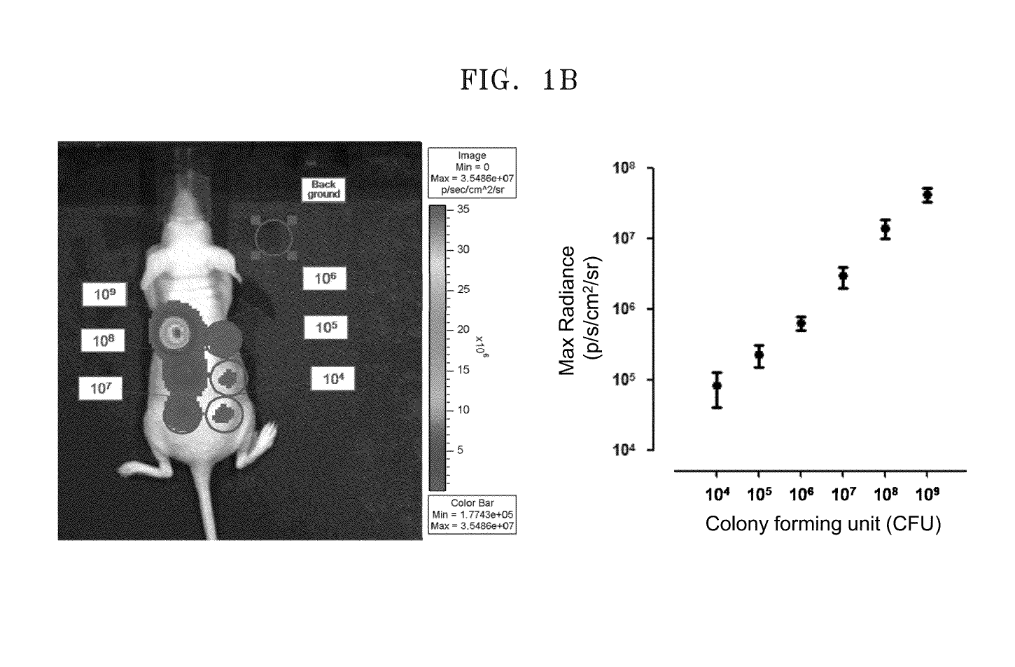

FIG. 1A illustrates results of (a) in vitro and (b) in vivo bioluminescence imaging of the SHJ2168 strain using the in vivo imaging system (IVIS) 100 imaging system (Caliper Life Sciences). FIG. 1A (a) illustrates results of incubating the SHJ2168 strain in a Luria-Bertani (LB) broth (left) and in a LB agar plate (right) at 37° C. FIG. 1A (b) illustrates results of subcutaneous injection of a culture of the SHJ2168 strain resuspended and serially diluted in 100 μl of 1× phosphate buffered saline (PBS, Gibco/Invitrogen, Carlsbad, Calif.), into each region of interest (ROI) of the mice.

FIG. 1B illustrates a correlation between the amount of the SHJ2168 strain injected and the intensity of the detected signals. Imaging signal from each region was quantified in units of maximum photons per second per centimeter square per steradian (p/s/cm²/sr). As illustrated in the graph (right) in FIG. 1B, as the injected amount of the SHJ2168 strain increases, the intensity of the signals increases. Thus, it was confirmed that the intensity of the signals generated by luciferase increased in proportion to the amount of SHJ2168 injected.

1.2. Targeting of Salmonella Strain According to Age of Animal

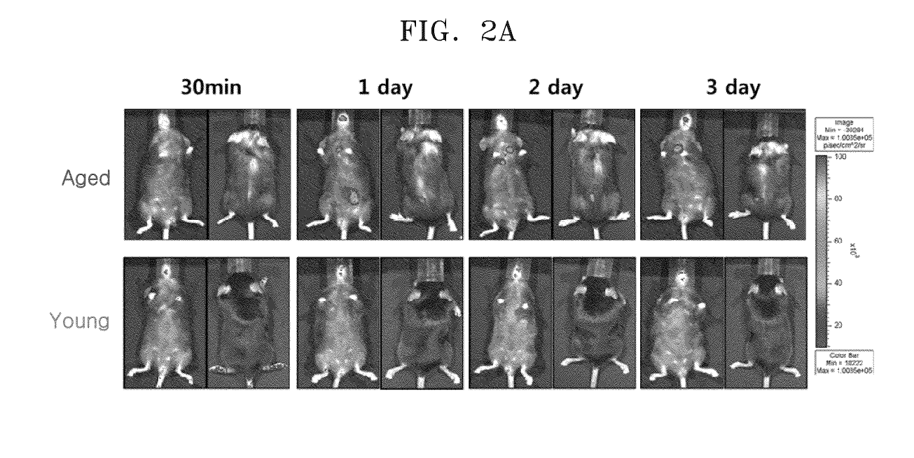

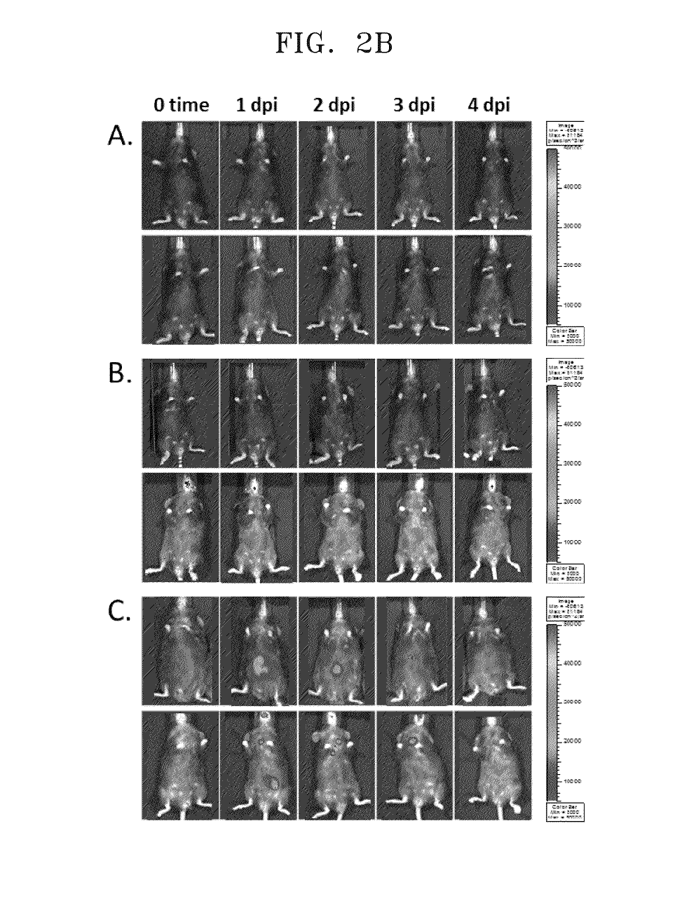

FIG. 2A illustrates images of targeting of the SHJ2168 strain injected into young and aged mice. Ventral (left) and dorsal (right) views of the same mouse at each time points are shown. 1×10⁷ CFU of the SHJ2168 strain in 100 μl of 1× PBS were injected into the lateral tail vein of 8-week old mice (bottom row, “young”) and 20-week old mice (top row, “aged”). Bioluminescence imaging was performed using the in vivo imaging system 100 (Caliper Life Sciences). Imaging signals were quantified in units of maximum photons per second per centimeter square per steradian (p/s/cm²/sr). At least 10⁴ CFU of bacteria produce a visible bioluminescence signal. At least 1×10⁵ p/s/cm²/sr of a signal intensity was considered to be significant. As illustrated in FIG. 2A, signals from luciferase were detected in 20-week old mice, while significant signals were not detected in the 8-week old mice.

FIG. 2B illustrates images of targeting of the SHJ2168 strain injected into mice with various ages. (A) to (C) respectively illustrate 2-month old mice, 12-month old mice, and 24-month old mice. 1×10⁷ CFU of the SHJ2168 strain in 100 μl of 1× PBS were injected into the lateral tail vein of the mice of each age. As illustrated in FIG. 2B, signals from luciferase were detected in thorax and/or abdomen of the 24-month old mice (Panel C), while signals were not detected in the 2-month old mice (Panel A) or 12-month old mice (Panel B). Intensity of the signals peaked at 1 or 2 days post injection (dpi) and was not detected at 4 dpi.

### Example 2: Examination of Organ Targeted by Salmonella Strain

1×10⁷ CFU of the SHJ2168 strain in 100 μl of 1× PBS were injected into the lateral tail vein of 8-week old mice (n=3) and 20-week old mice (n=14), and targeted organs were subject to follow-up examination from 24 hours to 72 hours.

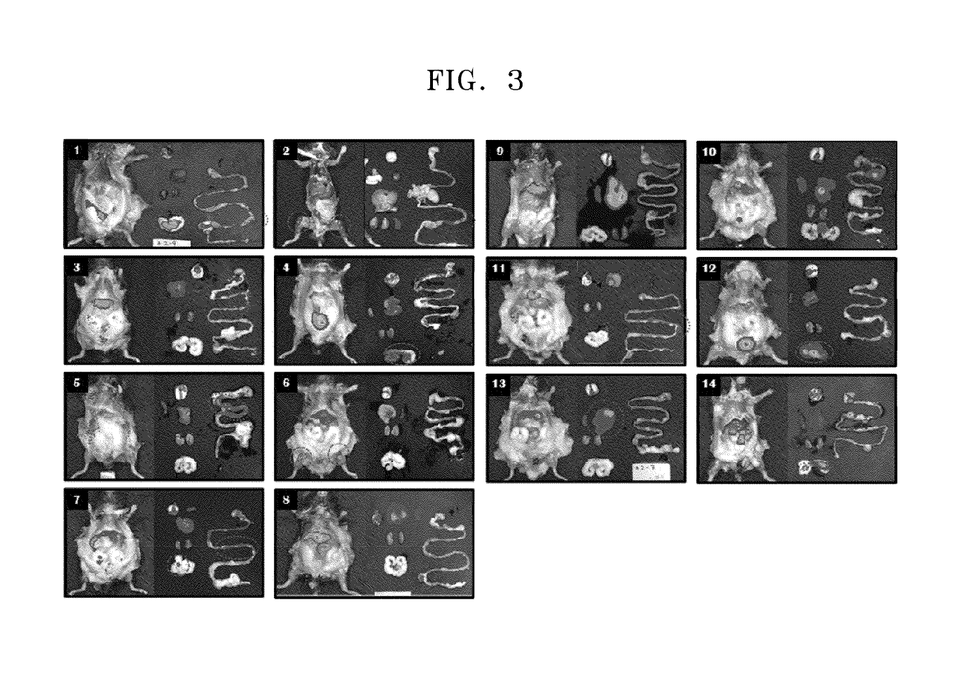

FIG. 3 illustrates images of targeting of the SHJ2168 strain injected into 20-month old mice. Significant luciferase signals were detected in all 20-month old mice except for one subject, with different distributions in the subjects. On the contrary, the signals were not detected in the 8-week old mice (data not shown).

Organs/tissues in which signals were detected are listed in Table 1 below.

TABLE 1

| Subject No. | Characteristics | Organ/tissue |
|---|---|---|
| 1 | | Intestine, semi vesicle |
| 2 | Osteoarthritis | Knee joint, distal intestine |
| 3 | | Intestine |
| 4 | | Intestine, urinary bladder |
| 5 | | Intestine |
| 6 | Osteoarthritis | Knee joint, liver |
| 7 | | Intestine, semi vesicle |
| 8 | | mesenteric lymph node |
| 9 | | mesenteric lymph node, liver |
| 10 | | Liver, semi vesicle, intestine |
| 11 | | Intestine |
| 12 | | Urinary bladder, semi vesicle |
| 13 | | Liver |
| 14 | | Liver, semi vesicle, intestine |

### Example 3: Analysis of Relationship Between Expression of Aging Marker and Targeting of Salmonella Strain.

1×10⁷ CFU of the SHJ2168 strain in 100 μl of 1× PBS were intravenously injected into 24-month old mice. The mice were euthanized two days after injection and processed for immunofluorescent imaging of endogenous p16 and γH2A in intestinal tissue sections. p16 and γH2A are markers of aged tissue because their expression has been shown to increase in tissues of aged mice (Lundberg et al., 2000, Curr Opin Cell Biol. 12(6):705-9; Wang et al., 2009, Aging Cell, 8(3): 311-323). Ileum was extracted from the mice and fixed overnight in 4% paraformaldehyde in PBS at room temperature. Fixed tissues were embedded in Optimal Cutting Temperature compound (OCT, Tissue-Tek) and frozen with a liquid nitrogen. The ileum was diagonally cut to sections having a thickness of 6 μm using a microtome-cryostat. The tissue sections were placed on a slide coated with aminopropyltriethoxysilane. The slide was washed with PBS (pH 7.4) to completely remove the OCT compound and incubated with mouse anti-Salmonella antibodies (1:100, Abcam) and either rabbit anti-p16 IgG antibodies (1:100 dilution; Santa Cruz Biotechnology) or rabbit anti-γH2A antibodies (1:100 Novus biological) overnight at 4° C. Alexa 488-goat anti-rabbit antibodies (1:100) and Alexa 568-goat anti-mouse antibodies (1:100) were used as secondary antibodies. Samples were immobilized with antifade reagent with DAPI (Invitrogen).

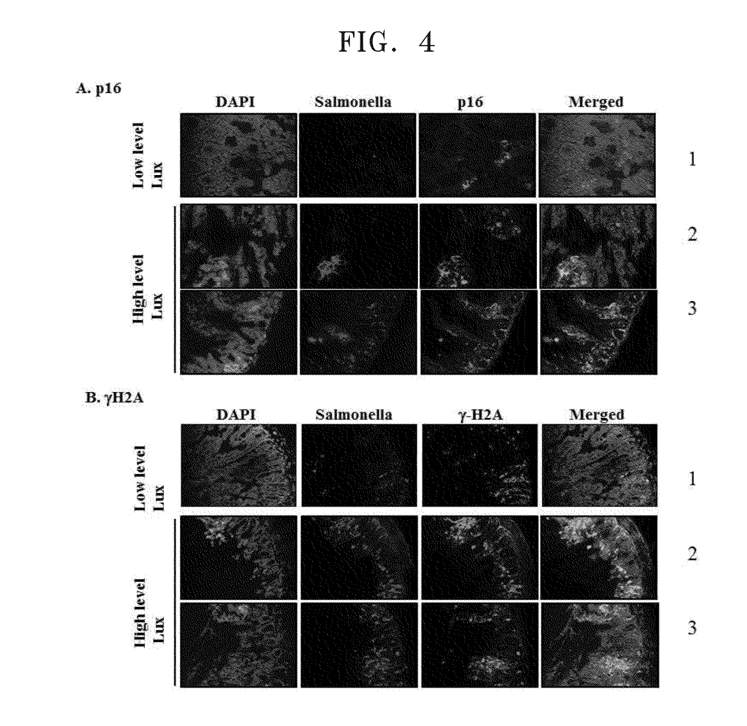

FIG. 4 illustrates the relationship between expression levels of p16 and γH2A and targeting of the SHJ2168 strain in the ileum of 24-month old mice. The SHJ2168 stain is expressed as red, and p16 and γH2A are expressed as green. Nuclei were stained using DAPI. The right-most column is a merged image. The panels labeled as ‘Low level lux’ represent regions where no or little lux signal is observed, while the panels labeled as ‘High level lux’ represent regions where lux signal is observed.

As illustrated in FIG. 4, positions where p16 or γH2A is expressed are nearly the same as those where the SHJ2168 strain accumulates.

As described above, according to one or more embodiments of the present invention, aged tissues may be detected by IVIS imaging of a mammalian subject subsequent to administration by injection of a recombinant bacterium comprising defective relA and/or spoT genes and further comprising a heterologous reporter gene comprising luciferase, a fluorescent protein, or the like.

It should be understood that the exemplary embodiments described herein should be considered in a descriptive sense only and not for purposes of limitation. Descriptions of features or aspects within each exemplary embodiment should typically be considered as available for other similar features or aspects in other exemplary embodiments. All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of detecting an aged tissue of a subject comprising:
administering to the subject a recombinant bacterium comprising
a mutation in a relA gene that reduces relA activity as compared to the same bacterium without the mutation,
a mutation in a spoT gene that reduces spoT activity as compared to the same bacterium without the mutation,
or any combination thereof,
and comprising a reporter gene;
and detecting position and/or intensity of a signal generated by the reporter gene in the subject, wherein position of the signal, or the position of the greatest intensity of the signal, corresponds to a location of aged tissue in a subject.

2. The method of claim 1, wherein the bacterium is Salmonella, Clostridium, or Escherichia genus.

3. The method of claim 1, wherein the subject is a mammal.

4. The method of claim 1, wherein the recombinant bacterium is administered to the subject intravenously.

5. The method of claim 1, wherein the reporter gene encodes a fluorescent protein or a luminescent protein.

6. The method of claim 1, wherein the detecting is performed by measuring fluorescence or luminescence.
